(19) United States
(12) Patent Application Publication
(10) Pub. No.: US 2026/0162771 A1
(43) Pub. Date: Jun. 11, 2026

(54) SPECIES CLASSIFICATION AND ORGANISM PREDICTION ENGINE

(71) Applicant: RTX BBN Technologies, Inc., Cambridge, MA (US)

(72) Inventors: Damianos Karakos, Cherry Hill, NJ (US); Bryan A. Bartley, Arlington, MA (US)

(21) Appl. No.: 19/410,136

(22) Filed: Dec. 5, 2025

Related U.S. Application Data
(60) Provisional application No. 63/728,401, filed on Dec. 5, 2024.

(51) Int. Cl. G16B 30/00; G06F 16/903

(52) U.S. Cl. CPC G16B 30/00; G06F 16/90335

(57) ABSTRACT

A method includes embedding a genomic sequence associated with a query organism into an embedding space which is pretrained based on labeled data associated with a plurality of training organisms. The method includes generating, based on embedding the genomic sequence into the embedding space, an explanation. The explanation includes an indication that the query organism is a reference organism included among a plurality of reference organisms. The explanation includes a description of one or more partial genomic sequences shared by the reference organism and the query organism.

### CROSS-REFERENCE TO RELATED APPLICATIONS

This application claims the benefit of priority to U.S. Application No. 63/728,401 filed Dec. 5, 2024, the disclosure of which is incorporated herein by reference in its entirety.

### BACKGROUND

Exemplary embodiments pertain to the art of synthetic biology and, in particular, to artificial intelligence (AI) systems that interpret biological sequencing data.

Synthetic biology has become a popular and impactful scientific field, as its findings can lead to profound benefits to society. On the other hand, there is potential for misuse: genome editing tools can be used to develop biological systems that can affect society in multiple negative ways, ranging from pandemics to biowarfare. For this reason, being able to quickly and accurately detect the presence of novel (especially engineered) organisms is of crucial importance.

### BRIEF DESCRIPTION

Disclosed is a species classification and organism prediction engine (also referred to herein as a classification and prediction engine). In an embodiment, the classification and prediction engine can be an AI system that organizes biological systems in a unique vector space, enabling the use of large-scale analytics tools for discovering novel associations, as well as quick and accurate detection of signs of biological engineering. Designed to work across a range of biological organisms that may be found in a variety of complex environments, such as metagenomic samples for biosurveillance and biodefense, one of the features of the classification and prediction engine is the ability to alert users about the presence of harmful pathogens and thus help prevent potentially catastrophic situations.

Example embodiments of the present disclosure are directed to a method including: embedding a genomic sequence associated with a query organism into an embedding space which is pretrained based on labeled data associated with a plurality of training organisms; and generating, based on embedding the genomic sequence into the embedding space, an explanation including: an indication that the query organism is a reference organism included among a plurality of reference organisms; and a description of one or more partial genomic sequences shared by the reference organism and the query organism.

In any one or combination of the embodiments disclosed herein, the explanation includes a description of whether the query organism is a fungus, a virus, or a bacteria.

In any one or combination of the embodiments disclosed herein, the explanation includes a description of whether at least a portion of the query organism is genetically engineered.

In any one or combination of the embodiments disclosed herein, the method further includes embedding reference sequences of the plurality of reference organisms into the embedding space, wherein generating the explanation is further based on embedding the reference sequences into the embedding space.

In any one or combination of the embodiments disclosed herein, the explanation includes a description of how the genomic sequence differentiates the query organism from one or more other reference organisms included among the plurality of reference organisms.

In any one or combination of the embodiments disclosed herein, the method further includes: generating embedding distances with respect to the query organism and the plurality of reference organisms, based on embedding the genomic sequence into the embedding space, wherein generating the explanation is based on the embedding distances.

In any one or combination of the embodiments disclosed herein, the method further includes: generating a visualization of an embedding distance with respect to the query organism and one or more reference organisms among the plurality of reference organisms, based on embedding the genomic sequence into the embedding space.

In any one or combination of the embodiments disclosed herein: the visualization includes statistical data associated with one or more features shared by the query organism and the one or more reference organisms, and the statistical data indicates contributions of the one or more features with respect to embedding distances between the query organism and the one or more reference organisms.

In any one or combination of the embodiments disclosed herein, the embedding space includes organism embeddings pretrained on the labeled data associated with the plurality of training organisms.

In any one or combination of the embodiments disclosed herein, generating the explanation includes processing the embedding space into which the genomic sequence has been embedded.

In any one or combination of the embodiments disclosed herein, the method further includes: determining, based on embedding the genomic sequence into the embedding space: a pathogenicity associated with the query organism; and a risk level associated with the pathogenicity, wherein the explanation further includes a description of the pathogenicity and the risk level.

Example embodiments of the present disclosure are also directed to a system including: an embedding engine configured to embed a genomic sequence associated with a query organism into an embedding space which is pretrained based on labeled data associated with a plurality of training organisms; and a classification and prediction engine configured to generate, based on embedding the genomic sequence into the embedding space, an explanation including: an indication that the query organism is a reference organism included among a plurality of reference organisms; and a description of one or more partial genomic sequences shared by the reference organism and the query organism.

In any one or combination of the embodiments disclosed herein, the explanation includes a description of whether the query organism is a fungus, a virus, or a bacteria.

In any one or combination of the embodiments disclosed herein, the explanation includes a description of whether at least a portion of the query organism is genetically engineered.

In any one or combination of the embodiments disclosed herein, the embedding engine is further configured to embed the plurality of reference organisms into the embedding space.

In any one or combination of the embodiments disclosed herein, the explanation includes a description of how the genomic sequence differentiates the query organism from one or more other reference organisms included among the plurality of reference organisms.

In any one or combination of the embodiments disclosed herein, the embedding engine is further configured to generate embedding distances with respect to the query organism and the plurality of reference organisms, based on embedding the genomic sequence into the embedding space, wherein the classification and prediction engine is configured to generate the explanation based on the embedding distances.

In any one or combination of the embodiments disclosed herein, the classification and prediction engine is further configured to generate a visualization of an embedding distance with respect to the query organism and one or more reference organisms among the plurality of reference organisms, based on the embedding engine embedding the genomic sequence into the embedding space.

In any one or combination of the embodiments disclosed herein, the embedding space includes organism embeddings pretrained on the labeled data associated with the plurality of training organisms.

Example embodiments of the present disclosure are also directed to an apparatus including: a memory having computer readable instructions and one or more processors for executing the computer readable instructions, wherein the computer readable instructions, when executed by the one or more processors, cause the apparatus to: embed a genomic sequence associated with a query organism into an embedding space which is pretrained based on labeled data associated with a plurality of training organisms; and generate, based on embedding the genomic sequence into the embedding space, an explanation including: an indication that the query organism is a reference organism included among a plurality of reference organisms; and a description of one or more partial genomic sequences shared by the reference organism and the query organism.

### BRIEF DESCRIPTION OF THE SEVERAL VIEWS OF THE DRAWINGS

The following descriptions should not be considered limiting in any way. With reference to the accompanying drawings, like elements are numbered alike:

FIG. 1 illustrates an example system supportive of species classification and organism prediction in accordance with one or more embodiments of the present disclosure. FIG. 1 further illustrates a graphical depiction of an embedding space (also referred to herein as an organism space) in accordance with one or more embodiments of the present disclosure, in which genomic sequences are represented as continuous vectors.

FIG. 2 illustrates an example flowchart of a method in accordance with one or more embodiments of the present disclosure.

FIG. 3 shows a two-dimensional projection of the embeddings of 40 HIATUS documents with a well-trained embedding stylistic model.

FIG. 4 shows a waterfall type of plot in accordance with one or more embodiments of the present disclosure.

FIG. 5 is a block diagram of a distributed computer system, in which various aspects and functions discussed herein may be practiced.

FIG. 6 illustrates an example flowchart of a method in accordance with one or more embodiments of the present disclosure.

### DETAILED DESCRIPTION

A detailed description of one or more embodiments of the disclosed apparatus and method are presented herein by way of exemplification and not limitation with reference to the Figures.

Embodiments described herein provide features for mapping genome sequences into a continuous vector space (an embedding space described herein), such that related organisms are relatively close to each other in the space, while unrelated organisms are relatively far apart. A classification and prediction engine provided in accordance with one or more embodiments of the present disclosure may take advantage of deep neural network technology capable of creating a generalizable embedding space, which enables the few-shot detection/characterization of organisms (under sparsity). The classification and prediction engine applies a contrastive learning paradigm, which is based on the presence of a large variety of organisms in the training of the model. Contrastive learning forces the model to learn, in a data-driven way, what makes organisms distinct, even if their differences are small. Embodiments enable the classification and prediction engine to focus on those genomic features that contribute to the discriminability between organisms.

In embodiments, the classification and prediction engine may be applied to microbial species: bacteria, viruses, and fungi. The classification and prediction engine is implemented as a deep neural network transformer architecture and detection system that maps the genetic sequence of a microorganism into an “organism space” (e.g., embedding space 111 later described herein), allowing for the employment of data mining tools (such as clustering) for finding interesting associations in the data (e.g., “tree-of-life” hierarchical structures) and which can be scaled to very large datasets. Embodiments may include explanation of important sequence features, a matching organism or organisms, and visualization as final outcomes.

In accordance with one or more embodiments of the present disclosure, the deep neural network transformer architecture and detection system may operate in a few-shot detection scenario. For example, with just one example of a novel or unknown genome, the detection system can detect similar genomes with high accuracy. For an example case of in which the novel or unknown genome is possibly an engineered genome, the detection system can detect similarly-engineered genomes with high accuracy.

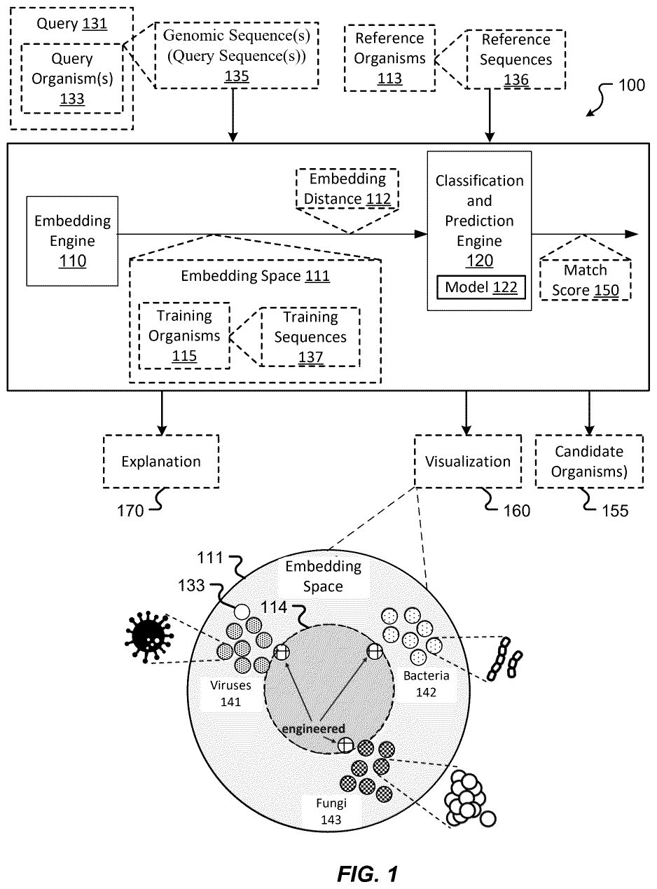

FIG. 1 illustrates an example of a system 100 that supports species classification and organism prediction in accordance with one or more embodiments of the present disclosure. The system 100 may include an embedding engine 110 and a classification and prediction engine 120. FIG. 1 further illustrates a graphical depiction of an embedding space 111 (organism space) provided by the system 100, in which genomic sequences are represented as continuous vectors. In the embedding space 111, related organisms are mapped closer to each other than unrelated organisms.

The classification and prediction engine 120 addresses the problems of challenges with scale, particularly in that the classification and prediction engine 120 provides features for handling challenging or sparse data sets for predictive models, including ecosystem-scale data across disparate databases. The classification and prediction engine 120 provides features for handling sparse datasets, such as few occurrences of a handful of engineered organisms (but not limited thereto), and using the sparse datasets in a fast and efficient manner for detecting other similarly-engineered organisms. For addressing the case of sparse datasets, the classification and prediction engine 120 may leverage contrasting information from other diverse species, from which enough samples are available. The classification and prediction engine 120 provides features for species classification and organism prediction for all kinds of organisms (i.e., naturally occurring and engineered, across sizes and complexities) by projecting the organisms into an embedding space 111 (a common organism space).

In one or more embodiments, the classification and prediction engine 120 can be implemented as an artificial intelligence (AI) system which organizes biological sequences in the embedding space 111 (an organism vector space), which the classification and prediction engine 120 may use for detecting previously unknown organisms (e.g., engineered, non-engineered), especially in sparse data situations.

Embodiments of the classification and prediction engine 120 may improve on current state-of-the-art (SOA) systems which have an inability to deal with highly recombinant sequences. Embodiments of the classification and prediction engine 120 may be incorporated as part of an ensemble scheme. In accordance with one or more embodiments of the present disclosure, the classification and prediction engine 120 provides classification and prediction techniques different from other technologies, and the classification and prediction engine 120 is trained on a large variety of publicly available resources (e.g., the NBCI Genome and the Sequence Read Archive (SRA), but not limited thereto). For example, the classification and prediction engine 120 may be trained on sequence reads.

Furthermore, the classification and prediction engine 120 provides a robustness to labeling errors, which is a serious problem in the bioengineering field. In some aspects, the classification and prediction engine 120 is data-driven. In some embodiments, the classification and prediction engine 120 may perform classification and prediction operations without a reliance on hand-crafted features or expert knowledge of biology or bioengineering (e.g., expert curated data). The classification and prediction engine 120 can automatically find the right features that discriminate between different organisms and species.

In accordance with one or more embodiments of the present disclosure, the classification and prediction engine 120 may include a trained model 122. The trained model 122 may be an AI model trained on various data (e.g., genome and sequence data). The trained model 122 may be trained based on relatively large datasets and be capable of understanding and processing such data. However, the trained model 122 is not limited to a model which is trained in a particular manner. In some embodiments, the trained model 122 may be a pretrained model and may be further tuned for example, with respect to improving performance.

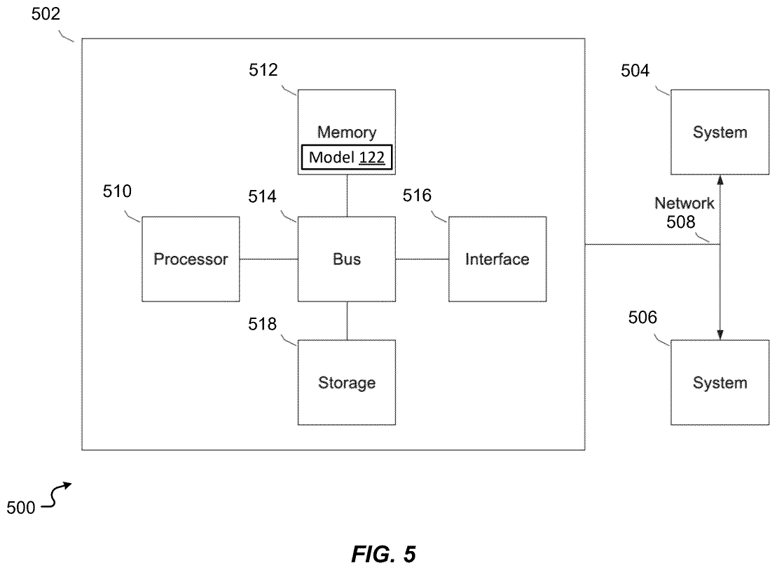

In some embodiments, the system 100 may be implemented in a computing system, example aspects of which are later described with reference to FIG. 5. In some other embodiments, the embedding engine 110 and the classification and prediction engine 120 may be integrated in the same computing device, or alternatively, in different respective computing devices capable of electronically communicating with one another.

As will be described herein, the system 100 may include software-executable code which, when executed by the system 100, may take a genomic sequence 135 (or a portion of the genomic sequence 135) of a query organism 133 included in a query 131 and identify the most likely organism from a pool of reference organisms 113 (i.e., potential matches). In some examples, the query 131 may include hundreds of thousands of query organisms 133, but is not limited thereto.

A query organism 133 included in the query 131 may also be referred to herein as an “unknown organism.” The genomic sequence 135 of the query organism 133 may also be referred to herein as a “query sequence.”

In some aspects, the system 100 may generate a match score 150 leading to attribution of a candidate organism 155 to the genomic sequence 135, a visualization 160 depicting the confidence with which the match score 150 leads to the attribution, and an explanation 170 of why the candidate organism 155 is more likely to be the match than other candidate organisms 155 (e.g., two or more other candidate organisms). In some embodiments, the system 100 may select the other candidate organisms 155 based on a random selection or other selection criteria.

Organism attribution as described herein is fundamentally a process of identifying, from a predefined set of reference organisms 113, a reference organism 113 whose reference sequence 136 corresponds to a given genomic sequence 135 of a query organism 133 included in the query 131. The attribution task may be framed within the context of a dataset which represents a collection of genomic sequences whose corresponding organisms are known. This is the training data. At implementation time, a genomic sequence 135 of a query organism 133 may be attributed to a known organism included among the reference organisms 113 by means of analysis implemented using the system 100 and techniques described herein.

In accordance with one or more embodiments of the present disclosure, the embedding space 111 may be trained with training sequences 137 (i.e., genomic sequences of training organisms 115) which are different from the reference sequences 136. In an example use case, the system 100 supports comparing a query organism 133 against a set of reference sequences 136 (e.g., engineered reference sequences, non-engineered reference sequences) which were not used in the training of the embedding space 111 and determining whether the query organism 133 matches any of the reference sequences 136.

Additionally, or alternatively, some of the reference sequences 136 may have been included among the training sequences 137 used for training the embedding space 111, but embodiments of the present disclosure are not limited thereto.

In the area of engineering detection, embodiments of the present disclosure are not limited to detecting whether a query organism 133 is an engineered organism or a non-engineered organism. The classification and prediction engine 120 may support features for detecting multiple kinds of engineering such as, for example, codon-optimization, gene insertion/assembly (i.e., diverse methods and mechanisms for editing sequences), plasmid cloning, and patchwork regions derived from different organisms suggestive of recombinatorial engineering.

Performance of the classification and prediction engine 120 may be measurable using false positive and false negative error rates. These can be combined into a single measure, equal-error-rate (EER), which is the point on the detection-error-tradeoff (DET) curve where the two errors are equal.

Embodiments of the system 100 described herein may lead to an increased and profound understanding of the microbial world. With the ability to map all organisms into a common vector space as provided by the system 100, the classification and prediction engine 120 provides learned deep neural models configured for discovering unique and not previously known associations between organisms, thus accelerating scientific discovery. The models described herein are capable of learning and improving models with more data.

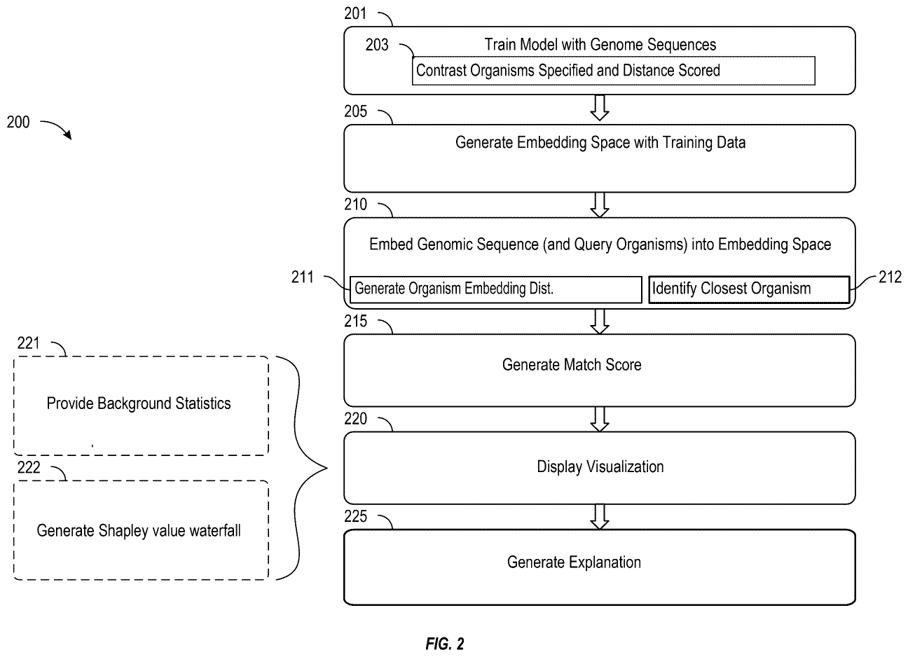

FIG. 2 illustrates an example flowchart of a method 200 supportive of species classification and organism prediction in accordance with one or more embodiments of the present disclosure. The method 200 may be implemented by the example aspects of the system 100 described herein. Aspects of the method 200 are described with reference to FIG. 1.

At block 201, the method 200 may include training a model with genome sequences. In some aspects, training the model at block 201 may include operations of specifying contrasting organisms and distance scoring (at block 203). For example, the contrasting organisms include training organisms 115 that are provided to train the model up front.

At block 203, distance scoring may include determining organism embedding distances 112 among the training organisms 115.

The contrasting training data used in training the model may include “hard-negative” samples. Hard-negative samples are contrasting pairs of sequences that are similar, but belong to distinctly different categories of interest. A non-limiting example of contrasting training data includes engineered E. coli versus non-engineered E. coli (or other host bacterium). Another non-limiting example of contrasting training data includes a pathogenic strain of E. coli versus a non-pathogenic strain of E. coli (or other such bacterium). In some cases, the contrasting organisms may be very similar genetically and have relatively small genetic differences, but the genetic differences can result in significant differences in terms of threat. Accordingly, for example, through the training of the model using contrasting organisms described herein, the model is capable of providing different respective threat classifications even for case of such small genetic differences.

At block 205, the method 200 may include generating an embedding space 111 with training data. The embedding space 111 may include organism embeddings pretrained on relatively large amount of labeled data (i.e. training data). The training data may include, for example, the training sequences 137 (i.e., genomic sequences) corresponding to the training organisms 115. The embedding space 111 may also be referred to as a pre-trained organism (embedding) space or an organism space.

As described herein, the embedding space 111 supports organism space mapping which enables engineered organism detection and pathogenicity level estimation. The embedding space 111 supports catalyzing novel detection for “unknown, unknowns” in biosurveillance, agnostic diagnostics, and invasive species detection.

In the embedding space 111, organisms having genomic sequences of a relatively higher level of similarity with respect to one another are relatively closer, and different organisms (i.e., organisms having genomic sequences of a relatively lower level of similarity with respect to one another) are relatively further apart from one another. For example, in the embedding space 111 illustrated in FIG. 1, viruses 141 are relatively closer to one another, bacteria 142 are relatively closer to one another, and fungi 143 are relatively closer to one another. Organisms relatively closer to the center of the embedding space 111 may represent engineered organisms.

In some examples, the level of similarity or difference may be based on traits of the genomic sequences, but is not limited thereto.

In some aspects, the embedding space 111 may be a pretrained embedding space. Accordingly, for example, embodiments of the present disclosure include training the embedding space 111 on labeled examples of a relatively large number of organisms (e.g., genomic sequences of millions of organisms). Embodiments of the present disclosure include forming the embedding space 111 by implementing a contrastive learning algorithm in which organisms with similar genomic sequences are made to be closer in the embedding space 111 and organisms with different genomic sequences are pushed further apart. Embodiments of the present disclosure include determining the similarity of genomic sequences by calculating the closeness of vector embeddings. In some embodiments, similarity may be based on Euclidean distance between vectors. In other embodiments, the calculation may be customized and tuned according to the user's notion or definition of similarity between training organisms 115.

In an example, the inputs used for training the embedding space 111 include genome sequences associated with known microorganisms from public databases. Aspects of an algorithm used for the pre-training of the embedding space 111 may involve contrastive learning through the use of “hard-negative” samples. Examples of contrasting categories include but are not limited to: engineered vs. non-engineered sequences and pathogenic versus non-pathogenic sequences. Contrastive learning forces the model to learn which features distinguish different classes of organisms, even if their differences are relatively small, and the techniques described herein may apply contrastive learning to tune the method for calculating similarity in order to maximize the distance in embedding space between contrasting pairs.

At block 210, the method 200 may include embedding a genomic sequence 135 of a query organism 133 into the embedding space 111. In some embodiments, at block 210, the method 200 may further include embedding query organisms 133 into the embedding space 111. In some embodiments, at block 210, the method 200 may further include embedding reference sequences 136 of the reference organisms 113 into the embedding space 111.

At block 211, the method 200 may include generating an organism embedding distance 112 with respect to a query organism 133 associated with the genomic sequence 135 and one or more of the reference organisms 113. The organism embedding distance 112 may be a numerical distance between the query organism 133 and the one or more reference organisms 113 in the embedding space 111.

Additionally, or alternatively, at block 211, the method 200 may include generating the organism embedding distance 112 with respect to each of one or more query organisms 133 and their associated genomic sequence or sequences. The organism embedding distance 112 may be a numerical distance between the query organism 133 and the one or more reference organisms 113 in the embedding space 111.

At block 212, the method 200 may include identifying a closest organism from among the reference organisms 113 with respect to the query organism 133. That is, for example, the method 200 may include identifying the closest organism based on the organism embedding distance 112. In the non-limiting example of FIG. 1, the system 100 may determine that the query organism 133 is a non-engineered virus.

In some aspects, at block 215, the method 200 may include generating a match score 150. In some aspects, the match score 150 may be inversely proportional to organism embedding distance 112 (e.g., a relatively lower organism embedding distance 112 corresponds to a relatively higher match score 150).

At block 220, the method 200 may include generating and displaying a visualization 160. In some embodiments, the visualization 160 may be a distribution graphic. In some aspects, the visualization 160 may illustrate a likelihood (e.g., probability) of whether a candidate organism 155 is an engineered organism. For example, a case in which respective organism embedding distances 112 between the query organisms 133 associated with the genomic sequences 135 are all relatively high (e.g., above a threshold value) may indicate that the genomic sequence 135 belongs to an engineered organism or that the query organism 133 was likely genetically engineered. In some other aspects, a case in which a representation of the query organism 133 in the embedding space 111 is located inside of a region 114 of the embedding space 111 may represent that the query organism 133 is an engineered organism.

In some aspects, at block 220, the method 200 may include depicting confidence with which a match score 150 leads to a given organism attribution. For example, the method 200 may include generating and outputting a confidence score associated with the match score 150.

The visualization 160 may include statistical data associated with the genomic sequence features shared by a known organism (i.e., as determined by the system 100 from among the reference organisms 113) and the query organism 133. For example, in generating and displaying the visualization 160, the method 200 may include (at block 221) providing background statistics with respect to the genomic sequence 135 of the query organism 133 in comparison to the genomic sequence of the known organism.

Additionally, or alternatively, in generating and displaying the visualization 160, the method 200 may include (at block 222) generating a Shapley value waterfall indicating what features of the genomic sequence 135 most contributed to a given organism attribution. The Shapely waterfall may indicate which features of the genomic sequence 135 contributed to the system 100 suggesting that a given candidate organism 155 included among the reference organisms 113 is the query organism 133. Shapley values may represent the contribution of a feature (e.g., a portion of the genomic sequence 135) to the output of an embedding distance model. Shapley values are a way of determining which features contribute most to a model, such as, for example, a model of similarity between organisms based on respective genomic sequences of the organisms.

At block 225, the method 200 may include generating an explanation 170 of why and how the reference sequences 136 are different or similar.

In a further example, for the case of species classification and organism prediction with respect to the query organism 133 associated with the genomic sequence 135, the system 100 may provide, in the explanation 170, a text-based explanation of why the query organism 133 is relatively close to a first reference organism or organisms 113 (e.g., a first virus, a first bacteria, or the like) in the embedding space 111 (i.e., the genomic sequence 135 is relatively similar to the reference sequence 136 of one or more reference organisms 113). For example, the explanation 170 may include examples of features or traits (e.g., based on respective genomic sequences) shared by the query organism 133 and the reference organism or organisms 113.

Additionally, or alternatively, the explanation 170 may include a text-based explanation of why the query organism 133 associated with the genomic sequence 135 is relatively far from a second reference organism or organisms 113 (e.g., a second virus, a second bacteria, or the like) in the embedding space 111 (i.e., the genomic sequence 135 is relatively different from the reference sequence 136 of the second query organism 130). For example, the explanation 170 may include examples of features or traits (e.g., based on respective genomic sequences) that distinguish the second reference organism or organisms 113 from the query organism 133.

Examples of the explanation 170 in accordance with one or more embodiments of the present disclosure is provided herein: The genomic sequence 135 may appear to be engineered by codon-optimization. The genomic sequence 135 may appear to have been edited via insertion of a synthetic gene. The genomic sequence 135 may appear to exhibit artifacts of molecular cloning. The genomic sequence 135 may appear to exhibit genetic features similar to known pathogens.

In some aspects, blocks 205 through 222 may be implemented by the embedding engine 110. In some aspects, block 225 may be implemented by the classification and prediction engine 120. That is, for example, the embedding engine 110 may generate the embedding space 111, and further, embed the genomic sequences 135 and query organisms 130 into the embedding space 111. The classification and prediction engine 120 (using the trained model 122) may generate the explanation 170 based on the embedding space 111, organism embedding distances 112, reference sequences 136, and match scores 150. However, embodiments of the present disclosure are not limited thereto, and aspects of the embedding engine 110 and the classification and prediction engine 120 may be implemented in a single engine capable of performing the described operations of both engines.

Embodiments of the species classification and organism prediction provided by the system 100 may include features and techniques provided by an authorship attribution system described in U.S. application Ser. No. 19/226,801, filed on Jun. 3, 2025, aspects of which are incorporated by reference. The authorship attribution system addresses (among other things) an authorship attribution problem and a machine text detection problem. In both of these problems, a piece of text is fed into the detector which outputs a decision as to which (known) author wrote it, or whether the piece of text was generated by a machine (e.g., a large language model (LLM)). Embodiments of the present disclosure include adapting features of the authorship attribution system to the biology domain, using, for example, a mapping such as in Table 1.

TABLE 1

| Authorship attribution system | Classification and prediction |
|---|---|
| (Explanations Engine) | engine 120 |
| Text document | Genetic sequence |
| Machine authored text | Engineered sequence |
| Multi-authored text | Recombinant sequence |
| Author ID | Organism |
| Authorship attribution | Classification of organism/species |
| Author profile | Engineering lab attribution |
| (e.g., country of origin) | |

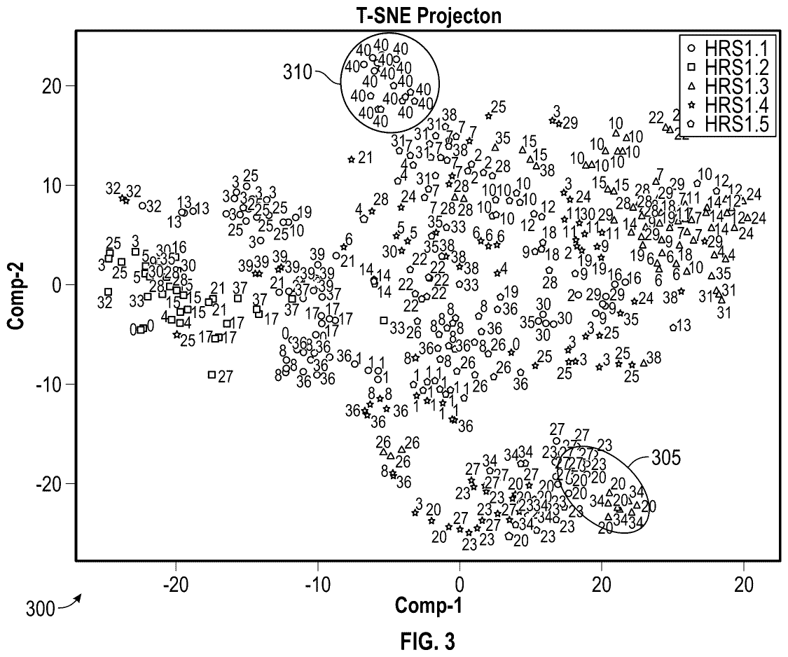

Given a text document, the authorship attribution system may apply a neural attention-based architecture to map the text document into an authorship embedding space. The training of the neural architecture is done with the criterion that, in the embedding space, data points corresponding to the same author are closer to each other than data points corresponding to different authors. FIG. 3 shows a graphical illustration of the embedding space provided by the authorship attribution system, where data points corresponding to the same author appear closer to each other in the learned space (bottom portion of FIG. 3) than in the unlearned space (top portion of FIG. 3), despite the fact that the same-authored documents are written in different genres. The approach follows work which uses contrastive learning for training, improved through the use of “hard-negative” samples. In the context of the classification and prediction engine 120, the genetic sequences will play the role of “documents” and the various labels given to them (e.g., national center for biotechnology information (NCBI) taxonomy ID) will play the role of “authors”.

Detection: At test time with respect to the authorship attribution system, the authorship attribution system decides if the embedding of the input (unknown) data is close to the embedding of one or more (few-shot) examples of: 1) writings of authors (authorship attribution); 2) writings of authors who write in a specific genre (genre detection); 3) machine-generated text (machine text detection); etc.

With reference to the system 100 and species classification and organism prediction applied to the biological domain in accordance with one or more embodiments of the present disclosure, the system 100 provides features for the detection of specific organisms, organism species, and/or engineered organisms. For example, non-limiting examples of questions the system 100 is capable of answering include: (a) Is the unknown sequence (e.g., genomic sequence 135) derived from virus, bacterium, fungus, or other? (b) Is the unknown sequence a signature of pathogenicity? What level of pathogenicity? What category of pathogenicity? and (c) Is the unknown sequence engineered or not?

Generalization to unseen authors and domains: A notable feature of the authorship attribution system on which the system 100 may be based is that the authorship attribution system can be applied to documents written in previously unseen genres and by previously unseen authors. None of the authors in the official evaluation data of the authorship attribution system were encountered in the corresponding training data. Despite this fact, the models of the authorship attribution system perform exceedingly well. This generalization of the models to unseen domains and authors supports effective predictions for new/unexpected situations in which the system has to make a prediction. The exposure of the model to a large variety of authors and domains during training makes it learn to pay attention to authorship characteristics that are largely domain independent.

With respect to species classification and organism prediction described herein, the model 122 may similarly be generalized to unseen domains and unseen organisms: in the few-shot detection scenario, the system 100 may compare the embedding of an unknown sequence (i.e., genomic sequence 135) with a handful of embeddings of other organisms (i.e., reference organisms 113). Based on the comparisons, the system 100 may make decisions on novelty, whether the unknown sequence is engineered or not (and, if yes, whether the engineering is novel), whether the unknown sequence is pathogenic or not, and/or determine the origin/attribution of engineering.

FIG. 3 shows a two-dimensional projection of the embeddings of 40 documents with a well-trained embedding stylistic model described with reference to the authorship attribution system. The numerical id of each author is shown next to the document/data-point he/she authored. Different patterns may be used to represent different genres. The red circles 305 and 310 show authors who appear close together in the trained embedding space, despite the fact that they have written in different genres. This kind of invariancy, to genre, is a notable aspect of the authorship attribution system.

Embodiments of the present disclosure support incorporating various types of invariancy with respect to species classification and organism prediction in the biology domain. For example, the system 100 may display, in the visualization 160 of the embedding space 111, organisms which appear far apart in the embedding space 111, despite the fact that the organisms exhibit genetic similarity as measured by conventional bioinformatic measures. As an example, for a query organism 133 that has been engineered, the system 100 may display the query organism 133 as far away in embedding space 111 from its non-engineered, naturally occurring progenitor species. As has been described herein, the system 100 provides an explanation component. The system 100 provides features for detecting similarities between organisms and further provides, in a human-understandable way, an explanation 170 for these similarities. In an example, the explanation 170 may indicate locations in the genome (or genomic sequence 135) where some kind of editing is evident (e.g., insertions, deletions, or the like)

In the context of species classification and organism prediction provided by the system 100 and the classification and prediction engine 120, the explanation 170 may take the form of base sequences that are common (or differ) between samples (e.g., two samples) being compared, and contribute most to the decision by the classification and prediction engine 120 (e.g., a candidate organism 155 as provided by the classification and prediction engine 120).

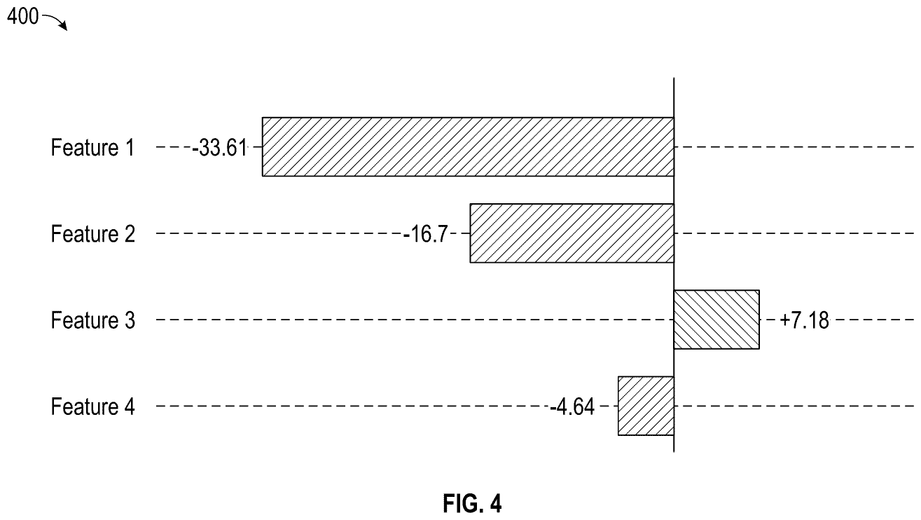

FIG. 4 shows a waterfall type of plot which may be generated by the system 100 in accordance with one or more embodiments of the present disclosure, containing the features (shown on the left) that have contributed most significantly (in terms of Shapley values, shown on the right) to an organism attribution decision by the system 100. Non-limiting examples of the features may include codon-optimization, gene insertion, artifacts of molecular cloning, sequences containing a patchwork of sequence regions suggesting recombinatorial engineering, or suspected virulence factors that may indicate an infectious organism.

As to risk mitigation, embodiments of the present disclosure may include implementing the classification and prediction engine 120 as a standalone component or as an additional component in such an ensemble scheme, offering a diverse approach to engineered organism detection for improving beyond other approaches.

For some cases, an engineered genome has too few or too isolated modifications compared to an original genome. Embodiments of the present disclosure provide increasing the amount of synthetic engineered data by 10×-100× in generating a trained model. Embodiments of the present disclosure provide engineering modifications including engineering artifacts and add these variations to the training data so that the trained model can learn to distinguish more types of engineering, even if they occur in isolated regions of the genome.

For some cases, raw sequencing may be too fragmented to identify distinguishing features. Embodiments of the present disclosure use annotated reference genomes in training, which allow us to reduce the raw data into shorter sequences of genes. Embodiments of the present disclosure may include splitting long genomic sequences into shorter fragments (e.g., genes), thereby adding granularity to embedding model.

As has been described herein, the systems and techniques described herein provide neural network training for continuous mapping to organism space, with a classification capability beyond other approaches. The systems and techniques described herein effectively apply mapped organism space to enable engineered organism detection. The systems and techniques described herein effectively apply a mapped organism space to enable pathogenicity level estimation tied to risk.

The systems and techniques described herein provide a deep neural net software system that maps genomic sequences into a continuous, organism embedding space, trained on sequence reads. Organisms mapped by the species classification and organism prediction techniques described herein are not limited to organisms known in advance. The systems and techniques described herein support few-shot detection: 1-2 samples of an organism enables detection of similar ones by measuring proximity in the embedding space. The systems and techniques described herein support adaptations to the biological domain: use of pretrained embeddings related to genomic bases, splitting genomic sequences into fragments (e.g., genes), and inclusion of protein sequences in addition to DNA.

In some embodiments, the systems and techniques described herein may significantly improve classification by emphasizing discriminatory factors. The systems and techniques described herein provide an embedding model trained by data from multiple genomic databases. The systems and techniques described herein further provide for data curation/formatting, embedding model training, and training a 3-way (e.g., bacteria, viruses, fungi) classification model on top of an embedding model, thereby providing an increased performance metric with respect to accuracy percentage. The systems and techniques described herein provide for training of a fine-granularity classification model (e.g., microbial families) on top of the embedding model.

In some aspects, the systems and techniques described herein overcome the inability of some other systems to identify distinguishing sequence features, thereby enabling bioengineered organism detection. The systems and techniques described herein provide for data curation for engineered organisms.

As has been described herein, the systems and techniques support artificial data generation for simulating engineered organisms. The systems and techniques described herein provide for retraining of the embedding model using both natural, engineered, and simulated organisms, thereby providing an increased performance metric with respect to weighted precision/recall.

The systems and techniques described herein provide features which introduce explainability to tie pathogenicity levels to risk without manual human analysis, thereby enabling a faster turnaround from data to decision. The systems and techniques described herein support effective data curation for organisms with pathogenicity levels. The systems and techniques described herein support retraining of the embedding model using the levels of pathogenicity. The systems and techniques described herein support effective training of the embedding model with hard-negative mining, focusing on hard-to-distinguish organisms.

FIG. 5 is a block diagram of a distributed computer system 500, in which various aspects and functions discussed herein may be practiced. The distributed computer system 500 may include one or more computer systems. For example, as illustrated, the distributed computer system 500 includes three computer systems 502, 504 and 506. As shown, the computer systems 502, 504 and 506 are interconnected by, and may exchange data through, a communication network 508. The network 508 may include any communication network through which computer systems may exchange data. To exchange data via the network 508, the computer systems 502, 504, and 506 and the network 508 may use various methods, protocols and standards including, among others, token ring, Ethernet, Wireless Ethernet, Bluetooth, radio signaling, infra-red signaling, TCP/IP, UDP, HTTP, FTP, SNMP, SMS, MMS, SS7, JSON, XML, REST, SOAP, CORBA IIOP, RMI, DCOM and Web Services.

According to some embodiments, the functions and operations discussed herein for species classification and organism prediction can be executed on computer systems 502, 504 and 506 individually and/or in combination. For example, the computer systems 502, 504, and 506 support, for example, participation in a collaborative network. In one alternative, a single computer system (e.g., 502) can be used to provide species classification and organism prediction according to the techniques described herein. The computer systems 502, 504 and 506 may include personal computing devices such as cellular telephones, smart phones, tablets, phablets, etc., and may also include desktop computers, laptop computers, etc.

Various aspects and functions in accordance with embodiments discussed herein may be implemented as specialized hardware or software executing in one or more computer systems including the computer system 502 shown in FIG. 5. In one or more embodiments, computer system 502 is a personal computing device specially configured to execute the processes and/or operations discussed herein. As depicted, the computer system 502 includes at least one processor 510 (e.g., a single core or a multi-core processor), a memory 512, a bus 514, input/output interfaces (e.g., 516) and storage 518. The processor 510, which may include one or more microprocessors or other types of controllers, can perform a series of instructions that manipulate data. As shown, the processor 510 is connected to other system components, including a memory 512, by an interconnection element (e.g., the bus 514).

The memory 512 and/or storage 518 may be used for storing programs and data during operation of the computer system 502. For example, the memory 512 may be a relatively high performance, volatile, random access memory such as a dynamic random access memory (DRAM) or static memory (SRAM). In addition, the memory 512 may include any device for storing data, such as a disk drive or other non-volatile storage device, such as flash memory, solid state, or phase-change memory (PCM). In further embodiments, the functions and operations discussed with respect to species classification and organism prediction can be embodied in an application that is executed on the computer system 502 from the memory 512 and/or the storage 518. For example, the application can be made available through an “app store” for download and/or purchase. Once installed or made available for execution, computer system 502 can be specially configured to execute the functions associated with species classification and organism prediction.

Computer system 502 also includes one or more interfaces 516 such as input devices (e.g., camera for capturing images), output devices and combination input/output devices. The interfaces 516 may receive input, provide output, or both. The storage 518 may include a computer-readable and computer-writeable nonvolatile storage medium in which instructions are stored that define a program to be executed by the processor. The storage 518 (storage system) also may include information that is recorded, on or in, the medium, and this information may be processed by the application. A medium that can be used with various embodiments may include, for example, optical disk, magnetic disk or flash memory, SSD, among others. Further, aspects and embodiments are not to a particular memory system or storage system.

In some embodiments, the computer system 502 may include an operating system that manages at least a portion of the hardware components (e.g., input/output devices, touch screens, cameras, etc.) included in computer system 502. One or more processors or controllers, such as processor 510, may execute an operating system which may be, among others, a Windows-based operating system (e.g., Windows NT, ME, XP, Vista, 7, 8, or RT) available from the Microsoft Corporation, an operating system available from Apple Computer (e.g., MAC OS, including System X), one of many Linux-based operating system distributions (for example, the Enterprise Linux operating system available from Red Hat Inc.), a Solaris operating system available from Oracle Corporation, or a UNIX operating systems available from various sources. Many other operating systems may be used, including operating systems designed for personal computing devices (e.g., iOS, Android, etc.) and embodiments are not limited to any particular operating system.

The processor and operating system together define a computing platform on which applications (e.g., “apps” available from an “app store”) may be executed. Additionally, various functions for generating and manipulating images may be implemented in a non-programmed environment (for example, documents created in HTML, XML or other format that, when viewed in a window of a browser program, render aspects of a graphical-user interface or perform other functions). Further, various embodiments in accord with aspects of the present disclosure may be implemented as programmed or non-programmed components, or any combination thereof. Various embodiments may be implemented in part as MATLAB or Python functions, scripts, and/or batch jobs. Thus, the disclosure is not limited to a specific programming language and any suitable programming language could also be used.

Although the computer system 502 is shown by way of example as one type of computer system upon which various functions for species classification and organism prediction may be practiced, aspects and embodiments are not limited to being implemented on the computer system, shown in FIG. 5. Various aspects and functions may be practiced on one or more computers or similar devices having different architectures or components than that shown in FIG. 5.

In some embodiments, the computer system 502 may be an edge computing system. For example, once the trained model 122 described herein has been trained, the model 122 is fairly lightweight (e.g., the computer system 502 may be implemented with a relatively low number of GPUs). Accordingly, for example, the computer system 502 may support remote monitoring for potential biothreats, DNA discovery in the remote environments (e.g., the ocean, space environments). The trained model 122 may be implemented, for example, as executable instructions stored in the memory 512.

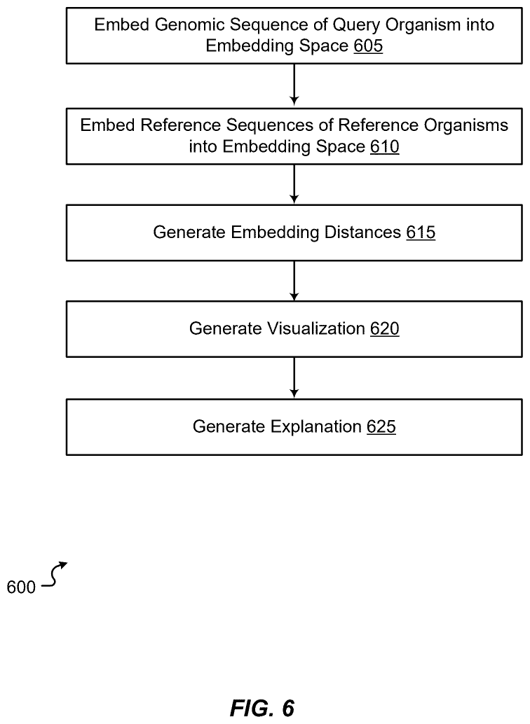

FIG. 6 illustrates an example flowchart of a method 600 in accordance with one or more embodiments of the present disclosure. The method 600 may be implemented by the example aspects of a system 100 described herein.

At block 605, the method 600 includes embedding a genomic sequence associated with a query organism into an embedding space which is pretrained based on labeled data associated with a plurality of training organisms. In an example, the embedding space includes organism embeddings pretrained on the labeled data associated with the plurality of training organisms.

At block 610, the method 600 includes embedding reference sequences (i.e., genomic sequences) of a plurality of reference organisms into the embedding space.

At block 615, the method 600 includes generating embedding distances with respect to the query organism and the plurality of reference organisms, based on embedding the genomic sequence (and the reference sequences) into the embedding space.

At block 620, the method 600 includes generating a visualization of an embedding distance with respect to the query organism and one or more reference organisms among the plurality of reference organisms, based on embedding the genomic sequence (and the reference sequences) into the embedding space.

In some aspects, the visualization includes statistical data associated with one or more features shared by the query organism and the one or more reference organisms, and the statistical data indicates contributions of the one or more features with respect to embedding distances between the query organism and the one or more reference organisms.

At block 625, the method 600 includes generating, based on embedding the genomic sequence (and the reference sequences) into the embedding space, an explanation including: an indication that the query organism is a reference organism included among the plurality of reference organisms; and a description of one or more partial genomic sequences shared by the reference organism and the query organism.

In some aspects, generating the explanation is based on the embedding distances.

In some aspects, the explanation includes a description of whether the query organism is a fungus, a virus, or a bacteria.

In some aspects, the explanation includes a description of whether at least a portion of the query organism is genetically engineered.

In some aspects, the explanation includes a description of how the genomic sequence differentiates the query organism from one or more other reference organisms included among the plurality of reference organisms.

In some aspects, generating the explanation includes processing the embedding space into which the genomic sequence has been embedded.

In some aspects, the method 600 may include determining, based on embedding the genomic sequence into the embedding space: a pathogenicity associated with the query organism; and a risk level associated with the pathogenicity. In an example, the explanation (generated at block 625) further includes a description of the pathogenicity and the risk level.

In the descriptions of the flowcharts herein, the operations may be performed in a different order than the order shown, or the operations may be performed in different orders or at different times. Certain operations may also be left out of the flowcharts, one or more operations may be repeated, or other operations may be added to the flowcharts.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A method comprising:
embedding a genomic sequence associated with a query organism into an embedding space which is pretrained based on labeled data associated with a plurality of training organisms; and
generating, based on embedding the genomic sequence into the embedding space, an explanation comprising:
an indication that the query organism is a reference organism included among a plurality of reference organisms; and
a description of one or more partial genomic sequences shared by the reference organism and the query organism.

2. The method of claim 1, wherein the explanation comprises a description of whether the query organism is a fungus, a virus, or a bacteria.

3. The method of claim 1, wherein the explanation comprises a description of whether at least a portion of the query organism is genetically engineered.

4. The method of claim 1, further comprising embedding reference sequences of the plurality of reference organisms into the embedding space, wherein generating the explanation is further based on embedding the reference sequences into the embedding space.

5. The method of claim 1, wherein the explanation comprises a description of how the genomic sequence differentiates the query organism from one or more other reference organisms comprised among the plurality of reference organisms.

6. The method of claim 1, further comprising:
generating embedding distances with respect to the query organism and the plurality of reference organisms, based on embedding the genomic sequence into the embedding space,
wherein generating the explanation is based on the embedding distances.

7. The method of claim 1, further comprising:
generating a visualization of an embedding distance with respect to the query organism and one or more reference organisms among the plurality of reference organisms, based on embedding the genomic sequence into the embedding space.

8. The method of claim 7, wherein:
the visualization comprises statistical data associated with one or more features shared by the query organism and the one or more reference organisms, and
the statistical data indicates contributions of the one or more features with respect to embedding distances between the query organism and the one or more reference organisms.

9. The method of claim 1, wherein the embedding space comprises organism embeddings pretrained on the labeled data associated with the plurality of training organisms.

10. The method of claim 1, wherein generating the explanation comprises processing the embedding space into which the genomic sequence has been embedded.

11. The method of claim 1, further comprising:
determining, based on embedding the genomic sequence into the embedding space:
a pathogenicity associated with the query organism; and
a risk level associated with the pathogenicity,
wherein the explanation further comprises a description of the pathogenicity and the risk level.

12. A system comprising:
an embedding engine configured to embed a genomic sequence associated with a query organism into an embedding space which is pretrained based on labeled data associated with a plurality of training organisms; and
a classification and prediction engine configured to generate, based on embedding the genomic sequence into the embedding space, an explanation comprising:
an indication that the query organism is a reference organism included among a plurality of reference organisms; and
a description of one or more partial genomic sequences shared by the reference organism and the query organism.

13. The system of claim 12, wherein the explanation comprises a description of whether the query organism is a fungus, a virus, or a bacteria.

14. The system of claim 12, wherein the explanation comprises a description of whether at least a portion of the query organism is genetically engineered.

15. The system of claim 12, wherein the embedding engine is further configured to embed the plurality of reference organisms into the embedding space.

16. The system of claim 12, wherein the explanation comprises a description of how the genomic sequence differentiates the query organism from one or more other reference organisms comprised among the plurality of reference organisms.

17. The system of claim 12, wherein the embedding engine is further configured to generate embedding distances with respect to the query organism and the plurality of reference organisms, based on embedding the genomic sequence into the embedding space,
wherein the classification and prediction engine is configured to generate the explanation based on the embedding distances.

18. The system of claim 12, wherein the classification and prediction engine is further configured to generate a visualization of an embedding distance with respect to the query organism and one or more reference organisms among the plurality of reference organisms, based on the embedding engine embedding the genomic sequence into the embedding space.

19. The system of claim 12, wherein the embedding space comprises organism embeddings pretrained on the labeled data associated with the plurality of training organisms.

20. An apparatus comprising:
a memory having computer readable instructions and one or more processors for executing the computer readable instructions, wherein the computer readable instructions, when executed by the one or more processors, cause the apparatus to:
embed a genomic sequence associated with a query organism into an embedding space which is pretrained based on labeled data associated with a plurality of training organisms; and
generate, based on embedding the genomic sequence into the embedding space, an explanation comprising:
an indication that the query organism is a reference organism included among a plurality of reference organisms; and
a description of one or more partial genomic sequences shared by the reference organism and the query organism.
